(19) United States
(12) Patent Application Publication
(10) Pub. No.: US 2022/0111119 A1
(43) Pub. Date: Apr. 14, 2022

(54) HOST MODULATING AND TISSUE REGENERATIVE HYDROGEL COMPOSITIONS AND METHODS OF THEIR DELIVERY TO PERIODONTAL AND PERI-IMPLANT TISSUES

(71) Applicant: Katalin C. Janosi-Fair, San Juan Capistrano, CA (US)

(72) Inventor: Katalin C. Janosi-Fair, San Juan Capistrano, CA (US)

(21) Appl. No.: 17/067,750

(22) Filed: Oct. 11, 2020

(51) Int. Cl. A61L 27/20; A61C 19/06; A61C 19/08; A61L 27/52; A61L 27/54; A61K 31/352

(52) U.S. Cl. CPC A61L 27/20; A61C 19/063; A61C 19/08; A61C 17/005; A61L 27/54; A61K 31/352; A61L 27/52

(57) ABSTRACT

The present invention concerns a novel use of therapeutic hydrogels in nonsurgical techniques to promote healing and regeneration of tissues whose structure and functions have been diminished due to chronic inflammatory disorders. More particularly, the invention provides therapeutic and regenerative hydrogel compositions and their nonsurgical delivery to periodontal treatment sites to treat periodontal and peri-implant diseases. Furthermore, the invention concerns a novel technique which involves the injection of said sterile therapeutic biomimetic hyaluronic acid hydrogel to which are tethered at least one host modulating agent, such as tetrahydrocurcuminoids, THC, and at least one additional tissue regenerative agent into the gingival connective tissue proper. The host modulating agent potentiates the periodontal tissue regeneration at the periodontal treatment site, while the biomimetic hyaluronic acid hydrogel serves in a dual capacity as a drug, a tissue regenerative agent carrier and as a biodegradable matrix around which the new connective tissue can regenerate. The invention also concerns oral care gel and paste compositions of at least one host modulating agent tethered to the HA tissue regenerative hydrogel formulated to be used in a synergistic home-based oral care regimen.

### RELATED APPLICATIONS

This application claims the benefit of U.S. Provisional Application No. 63/103,588 filed on Aug. 14, 2020.

### REFERENCES CITED

U.S. PATENT DOCUMENTS

| | | | |
|---|---|---|---|
| 0,279,043 A1 | September 2016 | Danenberg et al | A61K 8/447, 8/97 |
| 0,321,308 A1 | October 2019 | Majeed et al | A61K 31/12, 8/35 |
| 0,009,033 A1 | January 2020 | Gulizio | A61K 8/35, 8/9 789 |
| 6,123,957 | September 2000 | Jernbreg | 424/435 |
| 5,939,047 | August 1999 | Jernberg | A1K 9/50 |
| 10,413,637 | September 2019 | Bourdonet al | A61L 27/54, |
| | | | A61K 8/24 |
| 6,432,710 B1 | August 2013 | Boss et al | 435/366 |
| 10,449,214 B2 | October 2019 | Lennenmann | A61K 31/728 |
| 7,189,392 B1 | March 2007 | Kim et al | 424/94.1 |
| 0,009,033 A1 | January 2020 | Holdings | A61K 8/35 |

INTERNATIONAL PATENT DOCUMENTS

| | | | |
|---|---|---|---|
| WO 001325 A2 | January 2008 | Piramal et al | A1K 36/9066 |

### OTHER PUBLICATIONS

1. Deng, Z., Szafranski, S. P., Jarek, M. et al. Dysbiosis in chronic periodontitis: Key microbial players and interactions with the human host. Sci Rep 7, 3703 (2017). https://doi.org/10.1038/s41598-017-03804-8
2. Fan X, Alekseyenko A V, Wu J, Peters B A, Jacobs E J, Gapstur S M, et al. Human oral microbiome and prospective risk for pancreatic cancer: a population-based nested case-control study. Gut. (2016) 67:120-7. 10.1158/1538-7445.AM2016-4350 [PMC free article] [PubMed] [CrossRef] [Google Scholar]
3. Albandar et al., Manifestations of systemic diseases and conditions that affect the periodontal attachment apparatus: Case definitions and diagnostic considerations.
4. Lasserre J F, Brecx M C, Toma S. Oral Microbes, Biofilms and Their Role in Periodontal and Peri-Implant Diseases. Materials (Basel). 2018; 11(10):1802. Published 2018 Sep. 22. doi:10.3390/ma11101802
5. Panaro M. A., The emerging role of curcumin in the modulation of TLR-4 signaling pathway: Focus of neuroprotective and anti-rheumatic properties. Int J Mol Sci. 2020 April; 21(7): 2299
6. Litwiniuk M., et al. Hyaluronic acid in inflammation and tissue regeneration. Wounds. 2016; 28(3):78-88
7. Pober J S, Sessa W C. Inflammation and the blood microvascular system. Cold Spring Harb Perspect Biol. 2014; 7(1):a016345. Published 2014 Oct. 23 doi:10.1101/cshperspect.a016345
8. Battino M. et al., Oxidative injury and inflammatory periodontal diseases: The challenge of antioxidants to free radicals and reactive oxygen species, Crit Rev Oral Biol Med. 10(4): 458-476 (1999)
9. Franco C, Patricia H R, Timo S, Claudia B, Marcela H. Matrix Metalloproteinases as Regulators of Periodontal Inflammation. Int J Mol Sci. 2017:18(2):440. Published 2017 Feb. 17. doi:10.3390/ijms18020440
10. Caley M P, Martins V L, O'Toole E A. Metalloproteinases and Wound Healing. Adv Wound Care (New Rochelle). 2015; 4(4):225-234. doi:10.1089/wound.2014.0581
11. P. M. Preshaw, “Host modulation therapy with anti-inflammatory agents,” Periodontol 2000, vol. 76, no. 1, pp. 131-149, 2018. View at: Publisher Site|Google Scholar
12. Liang Y, Luan X, Liu X. Recent advances in periodontal regeneration: A biomaterial perspective. Bioact Mater. 2020:5(2):297-308. Published 2020 Feb. 28. doi:10.1016/j.bioactmat.2020.02.012,)
13. Citterio F, Gualini G, Fierravanti L, Aimetti M. Stem cells and periodontal regeneration: present and future. Plast Aesthet Res 2020; 7:41. http://dx.doi.org/10.20517/2347-9264.2020.29
14. Y. Ren et al, A collagen mimetic peptide-modified hyaluronic acid hydrogel system with enzymatically mediated degradation for mesenchymal stem cell differentiation. Materials Science and Engineering C 108 (2020) 110276
15. Misra S, Hascall V C, Markwald R R, Ghatak S. Interactions between Hyaluronan and Its Receptors (CD44, RHAMM) Regulate the Activities of Inflammation and Cancer. Front Immunol. 2015; 6:201. Published 2015 May 6. doi-10.3389/fimmu.2015.00201
16. Jiang D, Liang J, Noble P W. Hyaluronan as an immune regulator in human diseases. Physiol Rev. 2011; 91(1):221-264. doi:10.1152/physrev.00052.2009
17. Khunmanee S, Jeong Y, Park H. Crosslinking method of hyaluronic-based hydrogel for biomedical applications. J Tissue Eng. 2017; 8:2041731417726464. Published 2017 Sep. 6. doi:10.1177/2041731417726464
18. Bayer I S. Hyaluronic Acid and Controlled Release: A Review. Molecules. 2020; 25(11):2649. Published 2020 Jun. 6. doi:10.3390/molecules25112649
19. Trombino S, Servidio C. Curcio F. Cassano R. Strategies for Hyaluronic Acid-Based Hydrogel Design in Drug Delivery. Pharmaceutics. 2019; 11(8):407. Published 2019 Aug. 12. doi:10.3390/pharmaceutics11080407
20. Hershon L E. Elaboration of hyaluronidase and chondroitin sulfatase by microorganisms inhabiting the gingival sulcus: evaluation of a screening method for periodontal disease. J Periodontol. 1971; 42:34-36. [PubMed] [Google Scholar]
21. Vasconcelos A A, Pomin V H. The Sea as a Rich Source of Structurally Unique Glycosaminoglycans and Mimetics. Microorganisms. 2017; 5(3):51. Published 2017 Aug. 28. doi:10.3390/microorganisms5030051
22. Neuman M G, Nanau R M, Orufia-Sanchez L, Coto G. Hyaluronic acid and wound healing. J Pharm Sci. 2015; 18(1):53-60. [PubMed]

### FIELD OF THE INVENTION

The present invention concerns a novel use of therapeutic hydrogels in nonsurgical techniques to promote healing and regeneration of tissues whose structure and functions have been diminished due to chronic inflammatory disorders. More particularly, the invention provides therapeutic and regenerative hydrogel compositions and their nonsurgical delivery to periodontal treatment sites to treat periodontal and peri-implant diseases. Furthermore, the invention concerns a novel technique which involves the injection of said sterile therapeutic biomimetic hyaluronic acid hydrogel to which are tethered at least one host modulating agent, such as tetrahydrocurcuminoids, THC, and at least one additional tissue regenerative agent into the gingival connective tissue proper. The host modulating agent potentiates the periodontal tissue regeneration at the periodontal treatment site, while the biomimetic hyaluronic acid hydrogel serves in a dual capacity as a drug, a tissue regenerative agent carrier and as a biodegradable matrix around which the new connective tissue can regenerate. The invention also concerns oral care gel and paste compositions of at least one host modulating agent tethered to the HA tissue regenerative hydrogel formulated to be used in a synergistic home-based oral care regimen.

### BACKGROUND OF THE INVENTION

Periodontal disease, (PD) or gum disease is a common, chronic inflammatory condition of the oral cavity. If not halted, in time it results in tooth loss and debilitating edentulism due to a progressive degeneration of the periodontal supporting tissues, namely the connective tissues, followed by the attachment apparatus and the alveolar bone. There is increasing evidence that the gingival lesion initiated by a bacterial imbalance, called dysbiosis [1] if left untreated progresses toward tooth loss due to the host's prolonged and exacerbated inflammatory reaction to the persistent inflammatory triggers. Furthermore, peri-implant mucositis and peri-implantitis, the two conditions resulting in dental implant failure share a similar etiopathogenesis and pathophysiology with PD.

According to the Centers for Disease Control and Prevention (CDC), 47.2% or close to 1 in 2 adults in the U.S. aged 30 years and older have some form of PD. Periodontal disease increases with age, close to 2 out of 3 adults 65 years and older have this oral condition, with the highest prevalence rates of advanced periodontitis and tooth loss in minority or low socioeconomic subgroups with other underlying conditions. It is generally understood that currently there is neither effective cure for the inflammatory aspect of this disease and costly and painful surgical methods for regeneration of lost periodontal tissues is not achieved to a desired level. Furthermore, while dental implants are a viable replacement option for lost teeth, in patients with uncontrolled underlying conditions and/or uncontrolled periodontitis, there is a rise in implant failure as well.

Importantly, over the past two decades or so, periodontitis has been linked to other chronic inflammatory conditions such as cardiovascular disease, osteoarthritis, type II diabetes, low birthweight babies, acute pancreatitis, pancreatic cancer and recently Alzheimer's disease. The specific mechanisms underlying these links have not yet been worked out, but several hypotheses exist. Fan X., et al [2] provided recently yet more evidence corroborating the long-established microbial link. His study concluded that the same oral pathogens implicated in the initiation of PD may also confer a higher risk for pancreatic cancer via a bacterial carcinogenesis model. Furthermore, known PD pathogens, such as Porphyromonas gingivalis and Aggregatibacter actinomycemetcomitans, (formerly Actinobacillus a.) have been consistently cultured from infected heart valves, atherosclerotic plaques, and most recently from amyloid plaques of Alzheimer's patients. In addition, known risk factors for PD, such as ageing, type II diabetes, and recent findings that certain systemic medications and other age related chronic inflammatory conditions may impact PD, point to a hosts exacerbated inflammatory response as a strong bi-directional denominator. See Albandar et al., Manifestations of systemic diseases and conditions that affect the periodontal attachment apparatus: Case definitions and diagnostic considerations [3]. As evidence for a strong correlation between advanced PD and increased systemic inflammatory burden, plasma C-reactive protein levels, CRP, a common marker for systemic inflammation, have been shown to decrease in controlled clinical trials following traditional periodontal treatment. As it becomes evident that COVID-19 disease shares the same risk factors with PD, along with a common inflammatory pathophysiology, the question is raised about the possible role of untreated advanced periodontitis in the outcome of coronavirus infections. Accordingly, it would be desirable to provide compositions and methods to not only treat and regenerate lost tissues but would also support a healthy immune and inflammatory response.

The first stage of periodontal disease is gingivitis, which is diagnosed based on the classic observable signs of gingival inflammation, namely redness or erythema, swelling or edema, and tissue that bleeds easily when measured with a periodontal probe. In addition to these classic visual signs, the diagnostic criteria include measuring the small space formed by the free gingival margin as it folds inward to attach to the neck of the tooth at the cemento-enamel junction. In health this small space, called gingival sulcus measures 1-3 millimeters. Furthermore, the cause of gingivitis, namely the visible accumulation of a sticky whitish bacterial plaque, called biofilm is quantified. It is noteworthy to mention that this type of biofilm associated gingivitis can manifest as early as in childhood, and it always resolves within a day or two following professional treatment, namely mechanical removal of the biofilm. It was due to this observation made in the late 1950s, that the focus of professional periodontal treatment has ever since then been to reduce the bacterial burden. However, in patients with known risk factors, such as continued poor oral hygiene, smoking, metabolic, hormonal and/or immunological disorders, the gingivitis returns after a few weeks and over time it progresses from the initial lesion to a more established or chronic form. In this at-risk group of patients, if the chronic form of gingivitis is not treated in time, more specifically around age thirty or so, the lesion progresses and reaches the irreversible stage of periodontitis.

Periodontitis, the irreversible stage of PD is diagnosed by the deepening of the gingival sulcus due to gingival attachment damage and by a concomitant alveolar bone resorption evident radiographically. In addition to these irreversible changes, the gingival tissue proper continues to exhibit the classic signs of inflammation. Traditional treatment for periodontitis is also the mechanical removal of the biofilm and its calcified version, namely calculus from above and on the increasingly denuded root surfaces inside the periodontal pocket, called scaling and root planing. Recognizing that the bacterial biofilm shifts to an anaerobic complex of periodontopathogens in the deeper pockets, recently traditional treatment has been augmented with antimicrobials, such as the doxycycline based Arestin, or the chlorhexidine based Periochip placed inside pockets which measure 5 millimeters or more. In addition, as an adjunct to mechanical scaling and root planing, a broad spectrum antimicrobial subgingival irrigation, more specifically chlorhexidine gluconate is also available. However, while mechanical treatment results in complete, albeit at times temporary healing of the gingivitis lesion, scaling and root planing even when augmented with antimicrobials, is only effective in halting the progression of the periodontitis lesion and does not result in tissue repair or regeneration. What this means is that clinically the gingival tissue continues to manifest a low grade inflammation despite regular maintenance therapies and improved home care, and if there is a lapse in either aspect of maintenance, the disease usually recurs with further bone loss and progressive attachment loss. Additionally, in the presence of active periodontitis, osseo-integrated dental implants also become diseased.

It is now generally accepted that periodontal tissue degeneration is due to the host's exacerbated and prolonged inflammatory response to persistent inflammatory triggers, more specifically due to bacterial membrane breakdown products, called lipopolysaccharides, LPSs of gram-negative bacteria. The fields of bacteriology and immunology have greatly increased our understanding of cell surface receptors and signaling molecules. LPS binding to specific receptors, such as toll-like receptors, TLRs, in particular to TLR-4 on various host cells and directly to immunoglobulins is what triggers the complement, inflammatory and immune systems to release pro-inflammatory signaling proteins, more specifically cytokines and small molecular chemokines. It is noteworthy from the mechanistic perspective of the compositions presented in this application, that there is increasing evidence that both curcuminoids, their metabolically reduced metabolites, namely tetrahydrocurcuminoids, THCs and hyaluronic acid can bind to TLR-4 [5,6]. However, the downstream events that follow are very distinct depending on different substrate binding. While LPS binding to TLR-4 triggers proinflammatory events, THC and high molecular weight hyaluronic acid binding trigger anti-inflammatory events. Studies now confirm that it is due to chemokines and cytokines that the blood vessels dilate and become more permeable, resulting in erythema and edema of the gingival tissues which become increasingly infiltrated by host inflammatory and immune cells [7]. Histologic studies show that each stage of inflammation is characterized by a distinct cellular infiltrate. The acute phase, as in the initial lesion of gingivitis is characterized by polymorphonuclear, PMN or neutrophil infiltrate. Neutrophils are short lived and cause bacterial death by releasing myeloperoxidase, an oxidant antimicrobial into the extracellular matrix of the connective tissues. It is now apparent, that it is due to the release of these and other superoxide anion radicals, hydrogen peroxide, hydroxyl radicals, and reactive oxygen species, ROS that the oxidative stress of the ECM increases. Additionally, all these free radicals, FRs and ROSs are capable of damaging host cell membranes, associated biomolecules. Studies also show that not only do proteins denature or lose their tertiary folding in a high redox environment, but many of the ECM key molecular constituents like the polysaccharides and proteoglycans become fragmented. In addition to these structural proteins, there are many proteins in the ECM with important enzymatic functions. Cytokines are proteins and so are the transmembrane receptors. It is now evident that interaction of FR/ROS with these ECM enzymes, receptors result in changes in enzymatic activity and receptor inactivation. See Battino M. et al [8[. Inflammation, however, is a normal and necessary physiologic response due to various insults, such as trauma, burn, chemical, viral, or bacterial invasion, as in periodontal disease, and inflammation due to any of these insults progresses through the same stages. More specifically, acute inflammation is always followed by a third and stage, which is characterized by tissue infiltration of other types of immune cells, such as plasma cells, macrophages which can release more sophisticated proteases, namely matrix metalloproteinases, MMPs and other enzymes to digest the damaged tissue and bacterial toxins and products, so the healing and regeneration phases can ensue.

Under conditions of persistent bacterial burden; however, as in the case of untreated chronic gingivitis, the inflammatory response becomes exacerbated and prolonged. Chronic inflammation is characterized not only by an infiltrate of macrophages which can reside in the tissue for months, and other white blood cells such as T and B lymphocytes but there is evidence now that the resident fibroblasts and mesenchymal cells whose function is to maintain tissue health, become aberrantly upregulated. What this means is that while in health and normal turnover, fibroblasts secrete collagen and hyaluronic acid, major ECM components, in chronic inflammation, they secrete pro-inflammatory molecules and reduce their secretion of normal ECM building blocks. In fact, not only does the tissue lose its physiologic function, but also its physiologic resident cellular components. Additionally, studies show that during chronic inflammation, in addition to the high oxidative stress, there is also an increase in the release of proteases, hyaluronidase and matrix metalloproteinases, MMPs into the ECM. It is generally accepted now that the alveolar bone resorption, which characterizes chronic periodontitis is due to MMPs released not only by immune and inflammatory cells, but also by fibroblasts, bone cells, keratinocytes, and endothelial cells [9,10]. Given the critical role the host inflammatory response plays in periodontitis, extensive research has gone into finding host modulating agents as an adjunctive therapeutic modality for PD. Non-steroidal anti-inflammatories taken orally had more side effects than statistically significant benefit, and the only other host modulating agent used to date is the oral administration of a sub-antimicrobial amount, 20 mg twice/day of Doxycycline, Periostat for 30 days, and up to three months. Despite evidence that Doxycycline at this dosage modulates MMP activity, clinical studies did not prove that it resulted in statistically significant tissue healing and bone regeneration outcomes as compared to traditional treatments alone [11]. Hence, there continues to be a need for compositions that can downregulate the host's exacerbated inflammatory response and/or reduce the oxidative stress of the ECM, so that the next steps in physiologic tissue healing and regeneration can take place.

In response to this need, combined with a growing interest in the use of plant derived nutraceuticals or phytochemicals, various over the counter and in-office compositions designed to deliver antioxidants and anti-inflammatory agents to the periodontal treatment sites have been developed. For example, U.S. Pat. No. 0,279,043 A1 deals with a combination of N-acetyl cysteine, an antioxidant and antimicrobial and derivatives of cranberry polyphenols, also an antioxidant, that is delivered to the periodontal and peri-implant sites via a variety ways; namely, oral ingestion, topical application, or placement in the subgingival pocket. More recently, due to its longest track record as a potent systemic antioxidant and anti-inflammatory, a plethora of compositions are being developed incorporating the dietary spice curcumin in various compositions, ranging from toothpastes, gels, mouthwashes, etc. Recognizing the critical need to overcome the various patient compliance, bioavailability and retention issues of the previously mentioned methods, WO Patent No. 001325, addresses this by incorporating curcumin into a bio-adhesive composition. But the yellow spice curcumin, also used to dye fabrics and hair has the disadvantage that it stains not only porcelain, resin but teeth as well. Furthermore, its metabolite, tetrahydrocurcumin, THC which is not only non-staining, almost white, but it has increasingly been shown to be a more effective antioxidant. Recognizing these obvious advantages over curcumin, THC is increasingly incorporated in the more recent oral care compositions as disclosed in various patents. (U.S. Pat. Nos. 0,321,308 A1 and 0,009,033 A1 being the most recent.)

However, despite efforts to overcome these limitations of polyphenolic compositions, even non-staining THC compositions continue to suffer from bioavailability issues. Not only is the method of delivery, namely brushing, massaging or swishing highly reliant on patient compliance, but the effective amount that does get into the periodontal treatment site, has been shown to be quickly degraded, eliminated or diffuses back out through the permeable epithelium. U.S. Pat. No. 0,321,308 attempts to deal with this bioavailability issue by mixing THC with essential oils to better incorporate it into the various compositions. This; however, only solves the miscibility of THC—it still does not ensure its increased bioavailability or retention at the periodontal treatment site.

It is generally accepted that for a drug to be effective, a certain concentration level, namely, a certain therapeutic index must be maintained for a certain amount of time at the target location. Systemically administered drugs do not accomplish therapeutic objectives efficiently, and additionally they have potential toxic side effects. Hence, what is needed is an effective drug delivery system, more ideally, the local implantation of drug delivery systems to provide the effective amounts of antioxidants and anti-inflammatories, such as THC to the periodontal treatment sites that overcomes patient compliance issues, and the various shortcomings of compositions in the previous art. Additionally, a host modulating therapy even if it is delivered more effectively than in the methods and compositions in the previous art, would continue to have several limitations. Given that tissue regeneration and/repair is such an important aspect of definitive periodontitis and peri-implantitis treatment, ideally what is needed is a composition which would not only effectively treat the chronic inflammatory response but would also be regenerative.

The goal of definitive periodontal therapy has always been the regeneration of the periodontal tissues. Historically all such regenerative endeavors have fallen under the aegis of surgical periodontal treatments and have focused mainly on regenerating the attachment apparatus and the alveolar bone. There is increasing evidence; however, catapulting the functional roles of the connective tissue proper, namely the role of the gingival tissue, and more specifically the key role of hyaluronic acid as a cell-to-cell signaling molecule in health, disease, repair and regeneration. Despite this emerging information, the field of periodontal surgery continues to aim all its surgical tissue engineering efforts at the regeneration of the attachment apparatus and the alveolar bone. To that end various surgical strategies have been developed over the years. The common thread to these surgical techniques is the reflection of a periodontal flap and after thorough mechanical scaling of the root surfaces various regenerative compositions, biomaterials, growth factors, bone grafting materials, and recently even stem cells are applied and retained in place using a variety of membranes [12,13]. Regenerative surgeries are usually recommended for patients who do not respond to traditional non-surgical treatments, and for those patients who continue to have periodontal pockets which are 6 millimeters or deeper. While there have been a lot of exciting advancements in periodontal tissue engineering, surgical regenerative techniques continue to suffer from lack of predictability and outcomes which are not maximally satisfactory. There seems to be a consensus that what is needed is improved biomimetic scaffolds and a better understanding of the spatio-temporal orchestration of the physiologic healing repair and regeneration processes, so upon the surgical provision of the right signaling molecules and scaffolds, more predictable outcomes may be anticipated [12,13].

While historically the soft connective tissues of the body have been mostly recognized for their structural roles, just like the gingival connective tissue has been mainly appreciated for its role of connecting the teeth to the alveolar bone, recently the recognition of their various roles in health, disease and regeneration/repair have been intensifying. Now it is generally accepted that what accounts for these important functional roles of the connective tissue is the ubiquitous extracellular high molecular weight polysaccharide, namely hyaluronic acid, HA. Corroborating HA's important role in health and disease, clinical studies show that the plasma level of the enzyme which breaks down HA, namely hyaluronidase is increased not only in the other chronic degenerative inflammatory disease, osteoarthritis but also in periodontitis. Furthermore, due to its ability to bind to specific receptors of various host cells, it is now apparent that HA is the key cell-to-cell signaling molecule. Additionally, due to its anionic charge and ability to form crosslinked and porous gels, HA has been increasingly used to target specifically deliver therapeutic agents to treatment sites, as in cancer treatment.

HA's cell-to-cell signaling, and mainly drug tethering capabilities have been recognized in the past in various patents within the field of surgical periodontal regeneration and in nonsurgical periodontal therapy. For example, U.S. Pat. No. 6,123,957 deals with the surgical regeneration of lost tissues by delivering compositions comprising of a cellular recognition agent such as hyaluronic acid and a tissue regenerative agent to the periodontal treatment site via a traditional flap surgical technique. There are several limitations to the compositions and their delivery techniques disclosed in this patent. The HA and tissue regenerative composition is applied in a gel or paste form directly to the root surfaces in the traditional periodontal surgical flap technique. In this type of application, without a rigid membrane there is a clear lack of control of the material, which can easily move around or seep out from under the periodontal connective tissue flap, even if it is covered with a membrane. Furthermore, an inflammatory stage is an imminent part of healing from periodontal surgery, and the compositions of this patent do not provide anti-inflammatory agents. Without an anti-inflammatory agent, HA is not retained at the periodontal treatment site in a surgical approach and it is quickly degraded.

Realizing the need for an anti-inflammatory agent, U.S. Pat. No. 5,939,047, by the same inventor as the previous patent deals with a non-surgical delivery of an anti-inflammatory agent using esters of hyaluronic acid or crosslinked HA gels as drug carriers. While using a drug carrier system to deliver the necessary anti-inflammatories to the periodontal tissues improves retention and bioavailability, the method of delivery in this non-surgical application, namely brushing or rubbing the compositions into the tissues suffers from patient compliance and therefore bioavailability issues. Also, the anti-inflammatories used, namely non-steroidal anti-inflammatories or NSAIDs, not only suffer from the fact that they are not naturally derived and their continued use may have various side effects, but also newer natural phytochemicals, like THC are shown to act via a more varied range and periodontitis specific mechanisms as compared to NSAIDS. Furthermore, HA in this application is used merely as a drug delivery agent and not a tissue regenerative agent.

More recently, U.S. Pat. No. 10,449,214 B2 deals with the injection of a gel containing HA in a crosslinked form or a mixture of cross-linked and non-crosslinked forms into the periosteum of bone and periodontal ligament to regenerate bone or tissue surrounding bones. This patent also suffers from technical and compositional limitations. From a technical point of view, not only is it very painful, complicated, but it is also unnecessary to inject into the tiny space between the bone and periosteum. Because it causes a lot of post-operative pain, clinicians are warned against injecting near the periosteum while performing local anesthesia. To inject into such tight spaces also requires force that may result in needle breakage and/or blunting of the needle tip, causing unnecessary tissue trauma. Furthermore, where the HA is needed to perform its healing and regenerative roles is in the diseased gingival connective tissue. While HA has does have antioxidant and anti-inflammatory activities, when injected into inflamed tissue, it works better if the composition also contains an additional antioxidant and host modulating agent, to not only increase retention of HA, but also to potentiate its wound healing activity at the periodontal treatment site. Furthermore, it is becoming evident that the crosslinking of the hydrogel intended as a biodegradable matrix around which bone or tissue can proliferate and mature, needs to be cross-linked by an entirely different method than what is used for dermal fillers, as is in the published patent just referenced. More specifically, if the HA gel is cross-linked using inorganic compounds as is done in dermal fillers, it is not easily biodegraded and in areas where new bone or tissue regeneration is desired, it will not only delay that process but it will also increase the possibility of undesired scar tissue formation. See Ren et al. [14].

In summary, for the practicing clinician like the inventor, today there are neither definitive therapeutic compositions nor therapies available to effectively treat periodontitis and/or regenerate lost tissues. Because traditional non-surgical treatments merely halt the disease progression, because surgical periodontal therapies continue to be costly, painful and unpredictable, there is a need for cost-effective, non-surgical compositions and methods which result not only in more predictable tissue healing, regeneration but also disease remission and prevention. Accordingly, it would be desirable to provide not only compositions that are effective drug carriers of antioxidant and host modulating agents and have the potential to serve as a biomimetic healing matrix with additional alveolar bone and attachment regeneration potential, but also to provide nonsurgical in-office methods of the delivery of said compositions which overcomes patient compliance issues.

Furthermore, given that home care is such an essential component of treatment success, given that current over the counter compositions like toothpaste, gel and mouthwashes lack any evidence based anti-inflammatory, antioxidant and tissue regenerative activities, what is also needed in addition to the in-office treatment, are synergistic at-home therapeutic compositions which are formulated specifically for the gingival tissues in mind. Which, when used in a daily home care regimen not only enhance the efficacy of the in-office treatment but also reduce disease relapse due to systemic inflammatory impact.

### SUMMARY OF THE INVENTION

The present invention provides a comprehensive system to treat and prevent periodontal and peri-implant diseases by providing therapeutic hydrogel compositions with hyaluronic acid as the backbone, and methods of nonsurgical delivery of said compositions to periodontal lesions to heal and promote regeneration of structure and function of the various tissues lost due to the host's exacerbated and prolonged inflammatory response. More specifically, the present invention concerns a novel in-office therapy which involves a sterile injectable HA hydrogel composition tethered to which are at least one host modulating and at least one additional tissue regenerative agent and the injection of said hydrogel directly into the diseased gingival lesion either as an adjunct following traditional mechanical procedures or as a stand-alone therapy. Furthermore, the present invention also discloses various other embodiments of said host modulating and tissue regenerative hydrogel compositions to be used synergistically as the home oral care arm of this therapeutic and regenerative system. This synergistic therapeutic and regenerative compositions delivered in various non-surgical methods as part of a synergistic therapeutic and preventative system overcome the various shortcomings and limitations of the compositions and methods in the previous art as discussed below.

This invention is anchored not only in some well-established facts but also in several novel recognitions made by the inventor. What the profession had already known for more than four decades, namely that the tissue damage in periodontitis is due to the deleterious effects of an exacerbated and prolonged host inflammatory response, is still very much a fact today. Therefore, there has been an intense quest for effective host modulating adjunctive therapies ever since that recognition. But the profession had also recognized that in addition to host modulating therapies, there was also a need to regenerate the tissues that had been lost due to the disease process, and hence tremendous effort has also gone into regenerative surgical procedures and sophisticated tissue engineering. But there seemed to be one major flaw to these two distinct philosophies working independently of each other, namely, that neither the disease model, nor the surgical regenerative model recognized the common ground between them. More specifically, they both failed to recognize the bridge, the common denominator that had always been there—they failed to recognize the critical role of the gingival connective tissue proper. In periodontitis it is the gingival connective tissue that becomes diseased, disrupted, and dysfunctional first, and only after the pathophysiologic process reaches a certain point of no return is when the attachment apparatus and the alveolar bone become irreversibly damaged Until that irreversible stage is reached, the gingival connective tissue can heal and regenerate from within. So, the first key recognition made by the inventor was that what is primarily affected by the disease process is the connective tissue. It had been apparent through decades worth of clinical observations that despite traditional mechanical and/or antimicrobial treatments, the gingival connective tissue never regains its pre-disease appearance or architecture after the irreversible stage of periodontitis sets in. While in health, the gingiva is pink, firm, and stippled, after the onset of chronic periodontitis, it continues to manifest all the classic signs of inflammation despite traditional treatment. It became apparent that to effectively treat the chronic inflammatory aspect of periodontitis lesion, the host modulating agent needs to be effectively delivered directly to the tissues where the disease process is taking place. It became apparent that the most predictable method was to directly inject the host modulating agent into the gingival connective tissue proper. The inventor, who is also a clinical dentist injects into the gingival connective tissue routinely to deliver a local anesthetic. Based on this recognition it became apparent that target specifically injecting a sterile host modulating solution into the gingival tissue, such as the potent anti-inflammatory and antioxidant like tetrahydrocurcumin would solve all the bioavailability problems in the previous art. Furthermore, adding a vasoconstrictor such as epinephrine and/or an anesthetic agent, would not only ensure increased retention of the host modulating agent at the periodontal treatment site, but would also ensure a pain-free administration. While this by itself would have been a great improvement over the host modulating compositions and methods of delivery in the previous an, it was the recognition that it would be even more ideal to add a tissue regenerative agent, which made the compositions as disclosed in this application possible.

Unlike the goals of traditional surgical periodontal tissue regeneration, as conceptualized in this application tissue regeneration refers to the regeneration of the disrupted and dysfunctional gingival connective tissue primarily, and only secondarily to the regeneration of the attachment apparatus and the alveolar bone. This paradigm shift again stems from the same recognition that guided the therapeutic goals of the host modulating aspect of the compositions and techniques of this invention. More specifically, in this classic example of how structure dictates function, it became apparent that the first step toward alveolar bone and attachment regeneration is by necessity the re-establishment of the structure and function of the disrupted and dysfunctional gingival connective tissue. Historically, the various soft tissues of the body, including the gingival connective tissue, have been appreciated mostly for their structural roles. The main structural role of the gingival connective tissue is to bridge, to connect the teeth to the alveolar bone by interfacing with these two hard surfaces via collagenous ligaments and the periosteum. Over the past few decades, however, research has increasingly been showing that in addition to its structural roles, connective tissues have critical roles in tissue homeostasis, disease, healing, and regeneration. Not merely as passive structures encasing the blood vessels or providing a scaffold for growth factors and resident cells which are responsible for the synthesis of the various matrix building blocks, but also as active participants. The fields of biochemistry, molecular biology and immunology have greatly enhanced our understanding over the past decades regarding the cell-to-cell signaling roles of the extracellular matrix, and more specifically that of one of its major constituents, namely hyaluronic acid and its ability to actively orchestrate the various process, such as cellular migration, proliferation and differentiation necessary for healing, repair and regeneration.

Today many of the cell-to-cell signaling mechanisms by which HA effects its various roles in health, inflammation, healing, and tissue repair/regeneration have been worked out. Research shows that most all human cells have transmembrane binding sites for HA, namely CD44 receptors [15,16]. Fibroblasts and mesenchymal cells are the main resident cells which synthesize HA, a straight chain linear polysaccharide which can be modified and crosslinked once extruded into the ECM. Studies show that just like alveolar bone, the connective tissue and the ECM also turn over daily. It is noteworthy, that during physiologic homeostasis, the innate enzyme, namely hyaluronidase is responsible for HA breakdown. Similarly, upon bacterial invasion, it is the bacterial hyaluronidase which breaks down HA of the ECM. Furthermore, HA and ECM proteins also begin to break down and become damaged due to free radicals and reactive oxygen species released by the hosts' inflammatory cells intended to kill the invading periodontopathogens. There is evidence now that low molecular weight fragments of HA also have signaling ability and they bind to the same CD44 receptors. However, while high molecular weight HA, HMWHA is anti-inflammatory, binding of LMWHA to CD44 receptors triggers a proinflammatory downstream cascade of events. Furthermore, studies show that LMWHA, just like LPS can also bind to toll-like-receptors, providing evidence that chronic tissue disruption as in inflammatory periodontitis, can also contribute to the prolonged and exacerbated host inflammatory state, just like a persistent bacterial load. Therefore, it becomes apparent that supplying HMWHA back into the periodontal tissues is necessary to end the prolonged inflammatory state, so that tissue healing, repair and regeneration can begin. While HMWHA is known to be also an anti-inflammatory and antioxidant, it became apparent that if it is injected into the inflamed gingival tissue by itself, without another anti-inflammatory agent, it would be quickly fragmented and degraded before it could affect its regenerative activities. It became, apparent that the best way to potentiate HA's regenerative potential was to combine it with a potent antioxidant and anti-inflammatory host modulating agent. It was the serendipitous discovery that HA is increasingly being used as the backbone of novel hydrogels not only to deliver therapeutic agents but also as biomimetic scaffolds in various tissue engineering applications, that made the therapeutic and tissue regenerative hydrogel compositions as disclosed in this application mostly possible [17,18,19].

Hyaluronic acid is a natural and ubiquitous constituent of all human tissues, and therefore it has a long history of use not only in cosmetic dermal fillers, in intra-articular injections indicated for the treatment of degenerative osteoarthritis, but also as an over the counter dietary supplement. While in the above mentioned applications, HA is used mostly for its physicochemical, structural and viscoelastic lubricating properties, as studies increasingly reveal its cell-to-cell signaling capabilities, HA is becoming an indispensable backbone of novel hydrogels used not only in the biopharmaceutical but also in tissue engineering applications. It is noteworthy, however, that these newer therapeutic and tissue regenerative uses demand a very distinct and tissue specific formulations of the hydrogel from the previous cosmetic and lubricating applications. Several factors in the synthesis of the hydrogel become critical, especially regarding the source, the nature and extent of crosslinking, immunogenicity of the hydrogel, to name a few. Historically, HA has been obtained from chicken combs, but more recently it is being derived via bacterial synthesis. To ensure low immunogenicity, it is important that the HA used in the compositions disclosed in this application be of non-animal source. Furthermore, the type and concentration of crosslinked HA is also significant. Traditionally, HA used in dermal fillers have been crosslinked with inorganic compounds, such as glutaraldehyde. This type of crosslinking resists gel degradation, which ensures that the filler stays within the tissue for an extended period. When it comes to the use of hydrogels in tissue engineering, it has been found that this type of crosslinking is not beneficial. Not only does it eliminate the necessary biologically active sites for drug binding, but it is also detrimental to the degradation and remodeling of the ECM [14]. In the formulations of the hydrogel compositions disclosed in this application, all these various factors have been considered. In this manner, the hydrogel compositions ad disclosed in this invention overcome all the shortcomings of the HA gel compositions in the previous art which lack these important considerations.

Hydrogels are three-dimensional, hydrated polymeric scaffolds formed by crosslinked hydrophilic polymers with a high affinity for water and biological fluids, capable of absorbing from 10% up to thousands of times their dry weight in water. In recent years, due to their unique properties such as biocompatibility, biodegradability, flexibility, softness, etc., hydrogels have been widely investigated for biomedical applications like cell therapy, tissue engineering, drug delivery, and diagnostics [19]. For example, a crosslinked hyaluronic acid hydrogel film loaded with anti-bacterial and anti-inflammatory drugs have been studied for wound dressing, keratin- or polyvinyl alcohol-based hydrogels as scaffolds for cell growth, PEG-based hydrogel (DEXTENZA®), recently approved by the Food and Drug Administration, as ophthalmic inserts, etc.[19], a collagen mimetic peptide-modified HA hydrogel system seeded with bone marrow mesenchymal stem cells designed for cartilage tissue repair [14]. While therapeutic and regenerative hydrogels have not been used in non-surgical periodontal treatments, given that hydrogels are biodegradable hygroscopic materials that absorb water, undergo rapid swelling, maintain three dimensional networks capable of reversible deformation, have been proven to deliver drugs effectively and target specifically, and can be modified in various ways to suit the desired goal of the therapeutic and tissue engineering applications, hydrogels seem to be the most ideal compositions not only to treat periodontitis, but also to regenerate the damaged tissues. While various natural and synthetic materials have been used in hydrogel preparations, hyaluronic acid is the material in the preferred embodiments disclosed in this invention due its high biocompatibility, low immunogenicity and cell-to-cell signaling capacity. Furthermore, while various host modulating agents can be used tethered to the HA hydrogel, in the embodiments disclosed in this invention, tetrahydrocurcuminoids and its reduced metabolites are the preferred host modulating agents for the following well-researched mechanistic reasons. While THC has been known to be a potent antioxidant, anti-inflammatory, recent evidence has shed light that one of the ways THC effects its anti-inflammatory activity is by binding to TLR-4. It appears that by competitively binding to these receptors, bacterial LPS is unable to bind and cause a downstream cascade of inflammatory events. In this respect, THC becomes a superior host modulating agent than even the previously used doxycycline which exerts it host modulating effect by interfering with destructive matrix metalloproteinases. This is because, THC can stop the inflammatory cascade even before it has a chance to start.

Therefore, in the preferred in-office sterile injectable hydrogel compositions the therapeutic antioxidant and host modulating agent is THC, and/or any other reduced metabolite of THC, namely di-, hexa, or octahydrocurcuminoids and the additional tissue regenerative agent is bone morphogenetic protein, insulin like growth factor, or other cellular agents such as stem cells, fibroblasts.

In the preferred in-office method, the therapeutic and regenerative hydrogel can be injected into the gingival tissues as an adjunctive treatment following mechanical scaling and root planing.

In yet another method, the injectable therapeutic and regenerative sterile hydrogel is injected into areas of the gingival tissue which continues to exhibit persistent signs of inflammation following traditional periodontal therapy. To the knowledge of the inventor this is the first time that an intra-gingival host modulating, and tissue regenerative injection is disclosed. This safe and non-invasive novel delivery of the therapeutic and regenerative composition overcomes the many shortcomings of the various surgical and non-surgical tissue regenerative methods in the previous art. Treatment success is greatly increased with this technique as there is greater control over the many complications associated with a surgical approach. There is also significantly less pain and down time following the non-invasive injection technique disclosed in this invention. This preferred method of the in-office closed delivery of said injectable compositions also overcomes the patient compliance limitations of the methods of delivery in the previous art.

In still another embodiment of this invention, both the host modulating and the tissue regenerative agents are combined in a sustained release powder, paste or gel form and placed in a subgingival manner in the 5 mm or deeper periodontal pockets for enhanced antioxidant, anti-inflammatory activity and periodontal tissue regeneration with sustained controlled release in the periodontal treatment site.

According to another embodiment, the oral composition of the invention is an oral topical composition of therapeutically effective concentration of THC and HA added to prophylaxis “gum health” paste ingredients to be used in in-office services and treatments.

Because periodontal treatment success hinges greatly on several patient centered variables, namely oral hygiene and the presence of systemic chronic inflammatory and hormonal, metabolic conditions, the necessity of a home oral care program which not only supports physiologic wound healing but also a healthy inflammatory system, became apparent. It was recognized that current over the counter oral care products, including toothpastes and mouthwashes do not support physiologic anti-inflammatory wound healing and may be contributing and/or exacerbating the dysbiosis. Hence, this need for novel “gum health” compositions comprising of at least one host modulating and at least one tissue regenerative agent, which can synergize with the treatment goals of professional therapy is what made the invention disclosed in this application partly possible.

In yet another embodiment of this invention, the host modulating agent and the tissue regenerative agent are combined in an over the counter gum health” paste or gel composition which is delivered to the periodontal tissues via a soft toothbrush, as part of a home care maintenance regimen to potentiate the efficacy of the in-office therapies.

The invention also encompasses various other embodiments such as mouth rinses, tooth powder, floss impregnated with essential oil of THC and HA, chewing gum, gummies etc. for take home and over the counter use to prevent, treat the inflammatory aspect of periodontal and peri-implant diseases and to regenerate damaged periodontal tissue.

One further advantage of the compositions and methods disclosed in this application is that to the knowledge of the inventor, this is the first time that an evidence based systematic therapeutic approach is utilized which serves the foundation for not only the novel in-office definitive treatment but also synergistically for a home-based maintenance regimen. This combination of an office-based and home supported maintenance holds the promise for increased compliance and improved treatment success rates.

Yet another advantage of the present invention is the novel synergistic combination of a potent host modulating agent tethered to a biomimetic HA hydrogel composition which increases the retention and bioavailability of the therapeutic host modulating agent at the periodontal treatment site as compared to compositions which lack an effective drug delivery system. In a synergistic manner, an effective amount of THC, can immediately potentiate the action of the tissue regenerative scaffold, for example, by reducing the oxidative stress, by scavenging free radicals via its known superior antioxidant activity. Given that there is now evidence that oxidative stress can also induce MMPs, by reducing the ROS/RFs, pro-inflammatory MMP activity can be immediately downregulated. In this symbiotic manner, high molecular weight HA delivered to the periodontal tissues escapes free radical damage and can immediately also serve as a “wound healing scaffold” in its various known and yet to be discovered roles. As the biodegradable scaffold is slowly degraded by innate hyaluronidase, the various additional tissue regenerative agents, such as growth factors, etc., can be slowly released into the increasingly anti-inflammatory pro wound healing microenvironment. Furthermore, the pro wound healing HA hydrogel matrix can provide the microenvironment necessary for the resident fibroblasts, mesenchymal cells, osteoblasts and cementoblasts to switch from the aberrant inflammatory to wound healing phenotypes.

One object of this invention is to provide a synergistic composition of host modulating and tissue regenerative agents in an injectable biomimetic hydrogel base and a method of delivery of said hydrogel compositions in a cost-effective and non-surgical means to promote maximal and predictable periodontal tissue healing and regeneration.

Yet another object is to provide an effective over the counter home care products which support and synergize with the antioxidant, anti-inflammatory and regenerative treatment goals of the compositions used in the professional treatments.

Another object is to reduce systemic inflammatory burden by providing a sustained release anti-inflammatory over the counter composition which when used on a daily basis can significantly impact the systemic inflammatory burden as can be monitored with plasma C reactive proteins, more effectively than an anti-inflammatory without a drug delivery system. More specifically, since THC has been shown to impact the systemic inflammatory burden, patients who also suffer from other chronic inflammatory conditions have a dual benefit via the direct absorption of THC across the oral mucosa and gingival tissue, bypassing the barriers that ingestion through the GI tract poses.

All the above and other characteristics and advantages of the invention will be further understood through the following illustrative and non-limiting description of embodiments thereof with reference to the appended drawings

### BRIEF DESCRIPTION OF THE DRAWINGS

FIGS. 1A through 1D are diagrammatic views illustrating the histopathologic changes of the three subdivisions of gingivitis leading to periodontitis. FIGS. 1A through 1 C, represent the initial, early, and established lesions of gingivitis, and FIG. 1D is an illustration of periodontitis.

FIG. 2A is a view illustrating the injection into the diseased periodontal connective tissue of a sterile viscoelastic liquid or gel composition of THC and HA. FIG. 2B is a diagram illustrating the delivery of a THC and HA gel, paste via a toothbrush as part of a home care regimen.

### DETAILED DESCRIPTION OF THE INVENTION

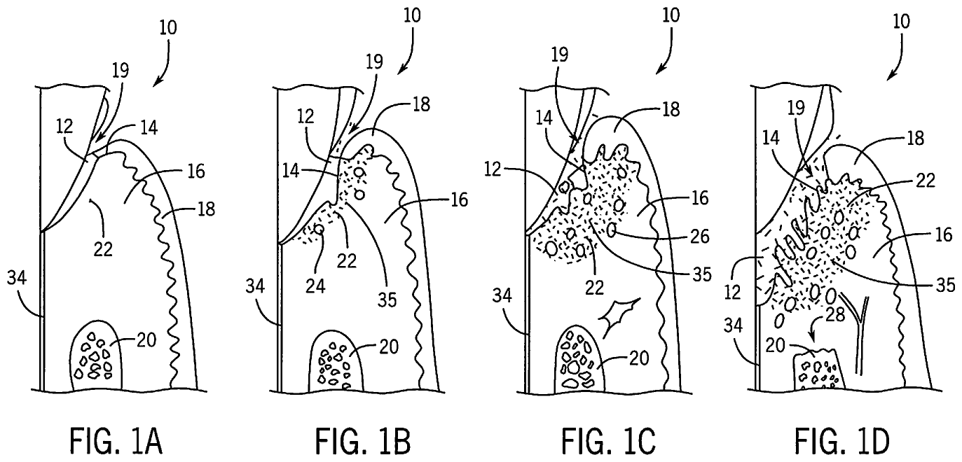

Referring to FIGS. 1A through 1D, which diagrammatically illustrate the human periodontal anatomy progressing from the initial lesion of gingivitis to periodontitis. Specifically, FIG. 1A illustrates the initial lesion of gingivitis which appears as an acute inflammatory response with neutrophil infiltration 22, vascular and epithelial 14, 18 and connective tissue 16 changes. The gingival epithelium 18 shows signs of inflammation; more specifically erythema and edema. The gingival connective tissue 16 is made up of a fibrillar collagenous network and an extracellular matrix composed mainly of hyaluronic acid, HA. There is evidence now that not only does the collagen denature due to free radicals, but so does HA. Furthermore, as immune, and inflammatory cells infiltrate the ECM, they migrate by the same mechanism that bacteria use, more specifically by releasing the enzyme, hyaluronidase. The alveolar bone crest 20, the junctional epithelium 12, with forms the attachment apparatus and the periodontal ligament 34 are undamaged. FIG. 1B illustrates the human periodontium afflicted with early gingivitis. The gingival tissue 16 shows signs of inflammation with lymphocytic infiltration, vasodilation, distension of the tissue, and increased spacing between the epithelial cells, including the gingival 18, sulcular 14 and junctional epithelial cells 12, leading due increased permeability. The sulcular epithelium, SE 14 becomes ulcerated and friable and the connective tissue 16 shows increased signs of collagen and ECM disruption 35. FIG. 1C is an illustration of the chronic or established lesion of gingivitis characterized by infiltration of monocytes 26 and continued CT destruction 35. There is evidence that in the chronic phase, in addition to free radicals and super-oxides, there is also a host of proteolytic enzymes, such as zinc dependent matrix metalloproteinases, or MMPs and a progression to T cell and B cell predominance 24. If treated, by the mechanical removal of biofilm and reduction of bacterial burden, the connective tissue 16 still has the capability to repair and regenerate itself without any permanent damage. FIG. 1D illustrates the human periodontium afflicted with periodontitis. The gingival tissues 16 are inflamed, the junctional epithelial 12 and the attachment apparatus have broken down due to both bacterial and host defense factors, i.e., A. actynomycetemcomitans, ROS, MMPs, etc. There is alveolar bone 20 resorption 28 and breakdown of the periodontal ligament 34 also due to the proteolytic activities of MMPs.

Broadly speaking, periodontitis is a chronic inflammatory disease which results in progressive periodontal tissue degeneration and tooth loss. As such, it is now apparent that to treat periodontitis what is needed in not only to effectively treat the disease but also to repair/regenerate the damaged tissues. More specifically, what is needed is a treatment modality which combines therapeutic and tissue regenerative agents, goals which traditionally have fallen under the separate aegis of non-surgical and surgical periodontics. Recent advances in non-surgical injectable hydrogel formulations which are able to not only deliver therapeutic drugs to treat the disease, carry growth factors and cells necessary to regenerate damaged tissues, but also serve as an immediate tissue regenerative scaffold around which the healthy connective tissue can reform, has finally bridged the gap between therapy and regeneration. It was the recognition by the inventor that what was needed to effectively treat the periodontal lesion was not only to treat but also to regenerate the diseased gingival connective tissue, furthermore the discovery of HA injectable hydrogels is what made the hydrogel compositions and methods of delivery to the periodontal tissues as disclosed in this application mostly possible.

It was also recognized that because the success of any professional periodontal therapy hinges greatly on a patient's home oral care regimen, there was a need for oral care compositions which synergize with the antioxidant, anti-inflammatory and tissue regenerative treatment goals of the injectable in office novel therapy as disclosed in this invention. A review of the existing over the counter oral care compositions revealed that none of the current compositions support the treatment goals of the compositions disclosed in this invention. It was this recognition which made the over the counter or home-based compositions for “gum health” as disclosed in this application possible. What is meant by the term “gum health” is a composition which is formulated specifically with ingredients which synergize and potentiate the antioxidant, anti-inflammatory goals of the in-office compositions as disclosed in this application. Furthermore, the “gum health” compositions as disclosed in this application will not contain any non-specific antimicrobials, or agents which have the potential to cause or exacerbate dysbiosis, such as non-nutritive sweeteners, except for xylitol.

Another critical factor in periodontal treatment success is the effective control of underlying systemic variables, such as other chronic systemic inflammatory conditions. It was the recognition that definitive periodontal treatment success also hinges on systemic conditions, such as cardiovascular disease, osteoarthritis, uncontrolled diabetes mellitus and others, that necessitated the home care compositions to also include an effective host modulating phytochemical with a known track record of systemic anti-inflammatory activity, such as THC.

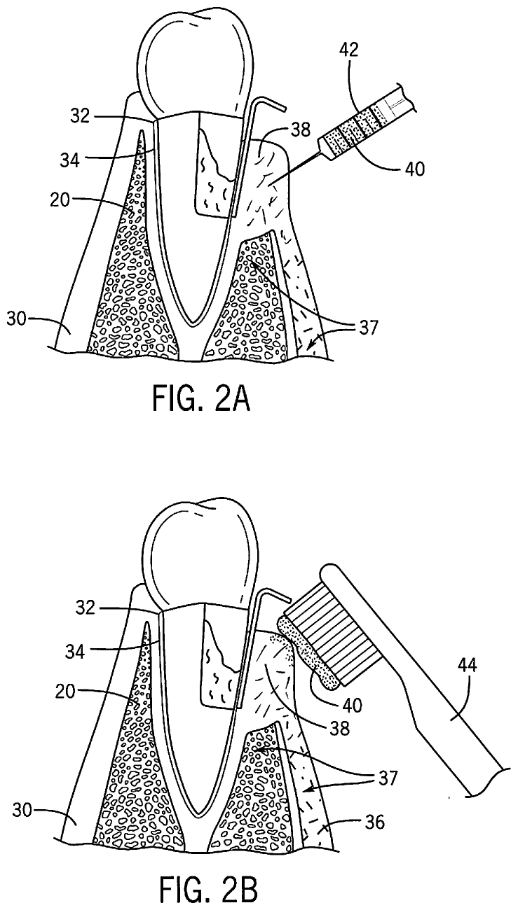

Accordingly, the present invention provides a systematic approach of methods and compositions for periodontal healing and regeneration of dysfunctional and/or lost periodontal tissue due to periodontal and peri-implant diseases. Specifically, in a first aspect, the present invention provides a method of treating periodontal and peri-implant disease and regenerating periodontal tissues by providing a sterile therapeutic biomimetic hydrogel composition with a hyaluronic acid backbone, tethered to which there is also at least one host modulating and antioxidant agent and at least one other tissue regenerative agent, and injecting the therapeutic and regenerative composition into the connective tissue of the periodontal treatment site as illustrated in FIG. 2A Wherein, the biodegradable hydrogel matrix mimics the physiologic granulation tissue providing an immediate scaffold around which the tissue can proliferative and mature resulting in definitive wound healing, as compared to compositions lacking an immediate biomimetic scaffold with an HA backbone. The host modulating agent effectively treats the inflammatory aspect of the disease and potentiates the tissue regenerative agent via a multiple mechanism as it will be illustrated in the remainder of the application. And the tissue regenerative agent works synergistically with the host modulating agent to effect maximal periodontal alveolar bone or attachment regeneration.

Various hydrogel preparations have been extensively documented in the patent literature and research studies. It is not the goal of this application to review all the various preparation modalities, but it is the goal to describe in detail the preparation modes selected to most ideally serve the novel periodontal specific goals and needs of the compositions disclosed herein. While injectable hydrogels have been used in many different applications in the field of therapeutic and regenerative medicine, to the knowledge of the inventor this is the first time that an injectable therapeutic and tissue regenerative hydrogel is used in periodontal and peri-implant therapy as disclosed in this invention. To develop a suitable hydrogel specific for the present application several factors were considered. More specifically, 1. the degradation rate and the mechanical properties of the hydrogel, which must complement the tissue growth and natural ECM of the periodontal tissues. These properties can be fine-tuned through variations in the chemical structure and density in the hydrogels which can be further controlled by choosing the type of crosslinking. Since HA is not able to form gels alone, several crosslinking methods have been considered. There are two basic types of crosslinking, namely, physical, or chemical. Nowadays, one of the most promising strategies for hydrogels preparation turns out to be click chemistry due to its high specificity, high yield, bio-orthogonality, and mild reaction conditions. Of the various click chemistry crosslinking, the Diels-Alder Reaction between furan modified HA and bismaleimide functional peptides seems to be appropriate for the application as disclosed in this invention due to the ability of this type of crosslinking to mimic the ECM. This type of crosslinking can further be subjected to thiol-ene photocoupling to allow its spatiotemporal patterning. Furthermore, the crosslinking can take place prior to injection or in situ, after the injection is delivered to the periodontal treatment sites. This can be accomplished via thermo-, photo, and/or pH sensitive hydrogels or an injection method with a double barrel syringe. There is ongoing research in quest of intelligent hydrogels with various features like stability, complex structures, biochemical cues, and responsiveness to several triggers. While certain crosslinking methods have been mentioned in the preparation of the HA hydrogels as disclosed in this invention, it is not meant to limit the scope of the possible preparation methods. It is understood within the scope of this invention, that as more advanced hydrogel preparations methods become available, they may also be utilized to prepare the hydrogel compositions as disclosed in this invention.

The material's physicochemical properties such as the degree of swelling and porosity must be chosen according to the needs of the therapeutic agents or cellular inclusions. Furthermore, Various materials can be used to form hydrogels; namely, natural, synthetic or hybrid. Due to their excellent biocompatibility, low immunogenicity the preferred material in the compositions in this application is a natural material that can be selected from the group of the following natural polymers: namely, hyaluronic acid, collagen/gelatin, chitosan, chondroitin sulfate, heparin sulfate, keratan sulfate, alginate, agar/agarose and fibrin, or a combination to form a composite or a hybrid. HA has been combined with both collagen and alginate to form hybrid hydrogels. Furthermore, HA can be derived from animal or non-animal sources. The preferred source of HA in this application is a non-animal source. Among biopolymers, hyaluronic acid (HA) represents one of the most used in the design of hydrogels for biomedical applications due to its biocompatibility, native biofunctionality, biodegradability, non-immunogenicity, cell-to-cell signaling ability, and versatility. HA-based hydrogels turn out to be versatile platforms ranging from passive and static matrices to smart, stimuli-responsive platforms with tunable properties and consequently they showed to have great potential as drug delivery systems, scaffolds for tissue engineering and regenerative medicine, and so on.

HA, whose chemically structure is depicted in the figure below, is a naturally occurring linear, non-sulfated glycosaminoglycan or polysaccharide that is widely distributed throughout the ECM of all connective tissues in humans and other animals. It is a GAG consisting of multiple repeating disaccharide units of N-acetyl-D-glucosamine and D-glucuronic acid. This polyanionic polymer has a range of molecular sizes from 1 to 10,000 kDa. HA is naturally degraded by hyaluronidase and oxidative species. Hyaluronidase has been shown to be elevated in the serum of patients diagnosed with periodontitis and osteoarthritis, providing indirect evidence that HA is depleted in the periodontal connective tissues of patients with chronic inflammatory periodontitis.

### FIG. 3: Chemical Structure of Hyaluronic Acid Polymer

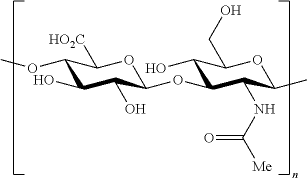

In the first aspect, the injectable therapeutic HA hydrogel is prepared using the Diels-Alder reaction as mentioned above according to the following steps. 1. Synthesis of furan functionalized HA. 2. Preparation of bismaleimide functional peptides. 3. Host modulating drugs are dissolved in water or ethanol. 4. Preparation of HA-furan drug loaded solution. 5. Drug loaded furan functionalized HA and bismaleimide functional peptides are mixed to form a hydrogel. All reactions performed at 37 C. 6. The hydrogel is sterilized prior to injection.

A variety of host modulating agents can be utilized in the compositions and methods of the present invention. According to one embodiment, the host modulating agent comprised within the combined composition of the invention may be selected from the group consisting of: Naturally derived polyphenolic phytochemicals, such as, non-staining tetra-, hexa-, octahydrocurcuminoids, or other non-staining polyphenols like cranberry extract, resveratrol, brown rice extract, biologics with immunomodulatory mechanisms as used currently in various other dermatological and chronic inflammatory bowel disease, arthritis, etc. Furthermore, other host modulating agents may also be selected from a variety of other herbs which have known TLR4 modulatory bioactivity. The following bioactive phytochemicals can be used in combination with THC: Parthenolide, a known inhibitor of the TLR4/NF-κB pathway, Berberine, an isoquinoline alkaloid mainly extracted from Rhizoma Coptidis, Sparstolonin B (SsnB) isolated from a Chinese herb (Sparganium stoloniferum) which was found to significantly inhibit the expression of the cytokines TNF-α, IL-6, and IL-1β induced by LPS. Atractylenolide I, a bioactive component of Rhizoma Atractylodis macrocephalae, significantly decreased LPS-induced TNF-α, IL-6, nuclear NF-kB.

In the preferred compositions, the host modulating agent is tetra-, hexa- or octahydrocurcuminoids, because these reduced forms of curcuminoids are known to have superior antioxidant bioactivity in addition to being a potent anti-inflammatory. THC also referred to as tetrahydrodiferuloylmethane, is the enzymatically reduced metabolite of curcumin, as shown in the Figure below.

### FIG. 4: Chemical Structures of Curcumin, DHC, THC and its Short-Lived Modified Metabolites

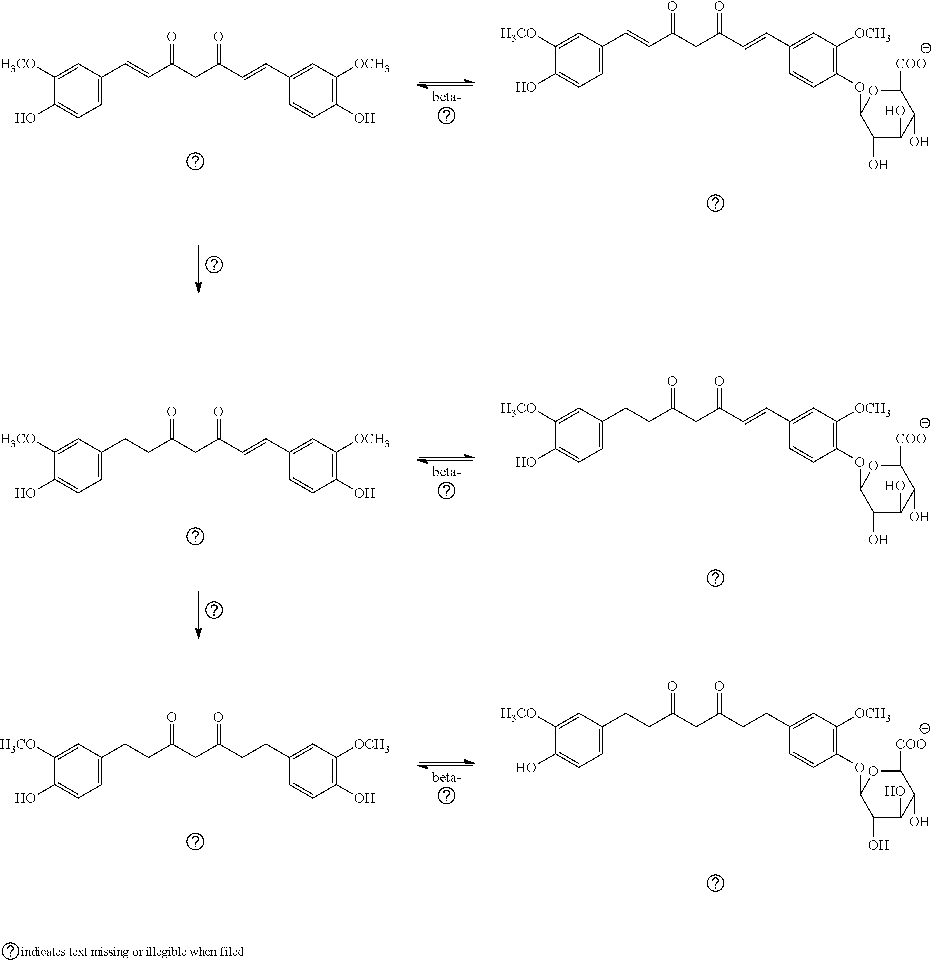

In addition to its well documented activities as an anti-oxidant, anti-inflammatory, hypoglycemic, and anti-cancer, curcumin, more specifically THC, has been shown to exert its beneficial effects on the molecular and cellular levels by modulating different signaling molecules, most likely via a Toll-like receptor binding, including transcription factors in gene expression, chemokines, cytokines, tumor suppressor genes, microRNAs, growth factors, cell cycle proteins, cell surface adhesion molecules and more. Moreover, there are emerging studies which substantiate THC wound healing efficacy. Its mechanism on the molecular level has been suggested by a recent study, which showed that this biphenolic molecule is able to mediate the switch of pro-inflammatory macrophage M1 to phenotype pro-healing macrophage M2 via Il-4 and -13. Furthermore, an in vivo study showed that by incorporating Il-4 in a gelatin nanofiber, resulted in resolution of periodontal inflammation and subsequent new alveolar bone generation. Furthermore, THC most likely also has antimicrobial efficacy, especially against anaerobic, gram negative bacteria.

In the preferred compositions, THCurcuminoids should be at least 96% pure and should have ideally the following concentrations, to mimic the composition in the rhizome. The preferred extraction method to result in the preferred composition is well documented in prior art.

Tetrahydro curcuminoids 90%
Tetrahydro demethoxy curcumin 9%
Tetrahydro bisdemethoxy curcumin 1%

The host modulating agent is supplied to the site of periodontal disease in an amount effective to facilitate or accelerate tissue healing via its antioxidant and anti-inflammatory properties. Typically, the therapeutic hydrogel composition contains an effective amount of host modulating agent in the range of about 0.001 wt. % to about 10 wt. % mg/ml, preferably about 0.05 wt. % to about 6 wt. %, preferably to about 0.01 wt. % to 4 wt. % l. For example, when the host modulating agent is THC, or hexa-, or octa-hydrocurcuminoids, the composition contains an effective among of THC, in the range of about 0.01 to 10 wt. % preferably from about 0.2 to about 6 wt. %, preferably about 5 wt. % relative to their dry macromolecular components.

In addition to the HA biomimetic hydrogel, at least one other tissue regenerative agent can be supplied to the periodontal tissue site to aid in the regeneration of alveolar bone and/or attachment apparatus by selecting from a group of bone morphogenetic proteins, growth factors, mesenchymal stem cells, fibroblasts, etc. Typically, the therapeutic hydrogel composition contains an effective amount of a tissue regenerative agent in the range of about 0.00001 mg/ml to about 99 mg/ml, preferably about 0.0004 to about 9.9 mg/ml, preferably about 0.001 mg/ml to about 5 mg/ml. For example, when the tissue regenerative agent is bone morphogenetic protein, BMP, the hydrogel composition contains an effective amount of BMP in the range of about 0.1 mg/ml to 99 mg/ml, preferably about 0.3 mg/ml to about 9 mg/ml, preferably about 0.2 mg/ml.

For example, when the tissue regenerative agent is mesenchymal stem cells, MSCs the hydrogel contains an effective among of MSCs in the range of about 0.1×10⁶ cells/ml, to about 10×10⁶ cells/ml, preferably 3×10⁶ cells/ml.

The compositions of the invention may further comprise certain antimicrobials, such as Doxycycline used not as a host modulating agent but as an antibacterial.

The composition of the invention may optionally further comprise at least one pharmaceutically acceptable carrier, diluent, excipient, vasoactive agent, anesthetic and/or additive in a physiologic buffer.

According to one aspect, the preferred embodiment provides a sterile HA hydrogel composition comprising of an effective amount of tetrahydrocurcuminoid powder, and at least one tissue regenerative compound, such as bone morphogenetic protein in a buffered physiological sodium chloride hydrogel, pH 7.2. Each 2.25 ml syringe will contain 0.001 wt. %-10 wt. % of non-staining THC powder, with sodium chloride 17 mg, disodium hydrogen phosphate 0.32 mg, sodium dihydrogen phosphate monohydrate 0.08 mg, a vasoactive agent such as epinephrine and water for injection q.s. to 2 ml. The composition of the invention may optionally further comprise of other compounds which have been shown to potentiate the efficacy of either the polyphenol or the tissue regenerative compound, and an acceptable carrier, diluent, excipient and/or additive. The contents of the syringe are sterile and nonpyrogenic.

According to the preferred method, this hydrogel composition is administered by a non-surgical intra-gingival injection using a 30-gauge hypodermic needle following mechanical periodontal therapy as illustrated in FIG. 2A. Injection is in small aliquots as instructed. In this preferred method, the injection of the present invention is performed following mechanical treatment such as scaling and root planing.

It will be appreciated that the therapeutic treatment compositions according to the composition and methods of the present invention can occur in any form. Specifically, the therapeutic treatment compositions can occur in a solid form, like a paste or gel, in a liquid form or combinations thereof. For example, in certain applications the therapeutic treatment composition can be comprised of a host modulating agent formed as a solid nano-particulates interspersed in a gel comprising of the tissue regenerative agent(s).

In a second aspect the invention provides “gum health” hydrogel and paste compositions comprised of a multi-action non-staining polyphenol, such and white tetrahydrocurcuminoids and at least one tissue regenerative compound, such as hyaluronic acid (HA) in known and effective amounts, 0.2% to 2% w/w of crosslinked and non-crosslinked HA intended to be applied to the gingival tissues as part of a home care regimen via a toothbrush as illustrated in FIG. 2B. The intended “gum health” compositions denote paste, gel, cream, spray, powder and/or liquid formulations. These oral care formulations are preferably brushed into the gingival tissue, but may also be rubbed into the gingival tissue, or swished around the oral cavity and after they are retained for an effective amount of time, they are expectorated. For increased efficacy, the patient is instructed to not eat or drink for at least thirty minutes after the application of the above compositions. Preferably the gum health composition is in the form of a paste or a gel.

The “gum health” compositions according to the invention will generally contain further ingredients but they will not contain any traditional toothpaste ingredients which are known to cause either allergic reactions, dysbiosis or an inflammatory response. While this is not a traditional toothpaste formulation, the gum health paste and gel compositions as disclosed in this application will in addition to wound healing bioactivity, be efficient in cleaning and strengthening the hard surfaces of the enamel.

The following is a list of the essential ingredients: 1. deionized water from about 2% to 45%, 2. humectant and rheology modifiers. Suitable humectants include xylitol, glycerin in the amount of 2% to about 55% respectively. 3. abrasives such as hydrated silica in the lowest effective amount from about 3 to 15%, 4. a thickening agent such as a natural gum like guar gum, carrageenan. 5. Natural flavorings such as mint, spearmint. 6. Sweeteners: Xylitol. 7. Calcium, phosphate minerals. The composition of the invention may optionally further comprise at least one other pharmaceutically acceptable antioxidant and anti-inflammatory active ingredient from the following list: Ascorbic acid, glutathione, tocopherol, retinoic acid or Vitamin A, zinc.

The following examples further describe and demonstrate embodiments within the scope of the present invention. These examples are given for the purpose of illustration only and may not be construed to limit the scope of the present invention. The ingredients can be mixed in any conventional manner. However, it has been determined that superior results are obtained when THC is pre-mixed with a mixture of crosslinked and non-crosslinked HA gel prior to incorporation with the remaining ingredients. HA used in this embodiment can be non-animal or animal source. Furthermore, the crosslinking in this application is not as specific as for the in-office injectable hydrogel compositions. Inorganic crosslinking with glutaraldehyde as used in the formulation of dermal fillers, intra-articular injections would be acceptable.

### EXAMPLES

### Example 1: Preferred “Gum Health” Paste Compositions

| Ingredients | % by weight |
|---|---|
| Tetrahydrocurcuminoids, THC | 6 |
| Hyaluronic acid, HA | 0.1-2 |
| Deionized water | 45 |
| Vegetable Glycerin | 30 |
| Hydrated Silica | 15 |
| Xantham Gum | 0.5-2 |
| Cocamidopropyl betaine | 0.5 |
| Natural Menthol | 2 |
| Tocopherol or Citric Acid | 0.5-2 |
| Walnut, coconut oil | 0.2 |
| Xylitol | 0.5-7.5% |
| Salt | 0.2 |
| Titanium dioxide | 0.05 |
| total | 100% |

While some of the mechanisms of the present invention rely on well-known and researched bioactivities of THC and HA separately, to the knowledge of the inventor, this is the first time that an antioxidant and anti-inflammatory host modulating agent is combined with a connective tissue regenerative agent in such a profound synergistic manner. Furthermore, as it will be disclosed in the remainder of this application, there are other mechanisms of bioactivity revealed in this invention with respect to both compounds which may not have been previously known or disclosed. It is due to the synergistic mechanisms of these two types of agents that the vicious cycle of chronic inflammation and tissue degeneration can be broken. The novelty of the invention does not rely merely on the compositions disclosed, but also on the novelty of the main delivery method of said compositions to the periodontal tissues, which overcomes the many shortcomings of delivery in the previous art of surgical periodontal tissue regeneration.

What is claimed is:

1. A method of healing and regenerating of periodontal and peri-implant tissues lost due to chronic inflammatory diseases comprising of the steps of:
a. Combining at least one host modulating agent and at least one tissue regenerative agent in a hyaluronic acid hydrogel base to form a therapeutic and tissue regenerative composition that is a sterile hydrogel or a sterile viscoelastic liquid, a nonsterile paste, a gel, a mouth rinse, or in a combination of these forms; the backbone of the hydrogel comprising of organically cross linked hyaluronic acid, or a mixture of non-crosslinked and crosslinked HA, a chemically modified hyaluronic acid, or a combination thereof, and
b. delivering the biomimetic therapeutic and tissue regenerative composition to a periodontal treatment site by injecting the sterile hydrogel or viscoelastic liquid into the gingival connective tissue as in an in-office treatment following traditional mechanical treatments, and/or applying the nonsterile therapeutic and tissue regenerative paste, gel, mouthwash by brushing, rubbing or rinsing as part of home therapy and/or maintenance. Wherein, the direct injection technique of the therapeutic and biomimetic tissue regenerative hydrogel into the gingival soft tissue ensures maximal bioavailability of both the host modulating agent and the tissue regenerative agent, and the hydrogel also serves as an immediate matrix around which the periodontal wound can heal from within much more effectively as compared to compositions lacking a host modulating agent or a drug carrier agent, or methods which do no ensure maximum bioavailability.

2. A method according to claim 1 wherein the first step includes the preparation of the biomimetic hydrogel or viscoelastic liquid using hyaluronic acid as the backbone. The non-animal hyaluronic acid can be crosslinked or in mixture of crosslinked and non-crosslinked from as dictated by the desired rheology of the composition. Preparation of the HA hydrogel composition should not follow the traditional inorganic method used in dermal fillers and intra-articular injections as documented in the previous art but should follow the use of organic crosslinking or the Diels-Alder reaction. It is not the aim of this invention to limit the methods of such hydrogel preparation. One criterion for the non-crosslinked form of HA in the hydrogel preparations used in this invention is that its molecular weight be between 750 kDa to 1400 kDa, preferably 1000 kDa.

3. A method according to claim 2, wherein the combining step includes selecting the therapeutic agent from a list of host modulating substances, such as non-staining phytochemical polyphenols or any other natural or synthetic host modulating agent with known antioxidant, anti-inflammatory and anti-microbial effectiveness, such as rice bran phenolic extract, or a biologic, or methylsulfonylmethane, MSM, or a combination thereof.

4. A method according to claim 3, wherein the host modulating agent is the reduced biologically active non-staining metabolite of curcumin, such as di-. tetra-, hexa-, octahydrocurcuminoids, or a combination thereof.

5. A method according to claim 4, wherein the combining step includes the addition of at least one additional tissue regenerative agent to the HA based hydrogel selected from a group of growth factors, glycosaminoglycans, collagen, bone morphogenetic protein, stem cells, fibroblasts or a combination thereof. While HA in the hydrogel is also tissue regenerative, adding an additional tissue regenerative agent further potentiates the efficacy of the hydrogel in regenerating the alveolar bone and the attachment apparatus in addition to the gingival connective tissue.

6. A method according to claim 5, wherein the sterilized biomimetic hydrogel comprising a therapeutic combination of a host modulating agent and at least one additional tissue regenerative agent is injected directly into already anesthetized diseased gingival connective tissue using a syringe, following traditional mechanical treatments using a 30 gauge needle, or needles of various sizes depending on the viscosity of the gel.

7. A method according to claim 5, wherein the sterile therapeutic biomimetic hydrogel also contains a vasoactive agent, such as epinephrine in concentrations of known amounts in a 7.2 physiologic phosphate buffered solution.

8. A method according to claim 5, wherein the sterile therapeutic biomimetic hydrogel also contains a local anesthetic, such as lidocaine, mepivacaine, etc. in a buffered solution.

9. A method according to claim 1, wherein the first step involves the preparation of the biomimetic and therapeutic paste or a gel which is delivered to the periodontal tissues using a toothbrush to heal and regenerate lost periodontal tissues due to inflammatory periodontal and peri-implant diseases as part of a home care regimen.

10. A method in claim 9, wherein the host modulating agent in the paste or gel compositions is THC, di-, hexa-, octahydrocurcuminoids or a combination thereof.

11. The method according to claim 10, wherein the composition comprises from 0.1% to 10%, preferably 6 wt-% of non-staining and colorless THC powder.

12. The method of claim 11, wherein additional tissue regenerative agent in the composition is non-crosslinked hyaluronic acid or a mixture of crosslinked and non-crosslinked HA with an average molecular weight of 1000 kDa and it comprises from 0.001 wt-% to 20 wt-%, preferably 0.2 wt-% of the composition.

13. A method in claim 12, wherein certain other excipients, humectants, rheology modifiers, low grade abrasives, flavoring agents, sweeteners may be added to the paste or gel compositions.

14. A method in claim 1 wherein the configuring steps include encapsulating at least of one the host modulating agents in microspheres which have time release values and adding to the paste embodiment as in claim 9 and delivering it into periodontal pockets which are 5 millimeters or deeper.

15. A method according to claim 14, wherein encapsulating comprises selecting a nanosphere having a time release value for substantially continuous release of the therapeutic treatment composition over a predetermined period.

16. A method according to claim 14, wherein combining comprises mixing the tissue regenerative agent with a polymer comprising the microspheres.

17. A method according to claim 13, wherein the therapeutic treatment composition is in a toothpaste, gel, or semisolid embodiment and it is applied to the disease periodontal tissue, preferably with a soft prophylaxis cup as part as part of an in-office treatment. The patient is instructed to refrain from eating or drinking for at least 30 minutes.

18. The method in claim 1, wherein the therapeutic composition is in the form of a mouthwash and it is delivered to the diseased periodontal tissues by rinsing with said liquid composition.

19. The method in claim 1, wherein the therapeutic composition is a chewing gum, or a gummi and it is delivered to the diseased periodontal tissue by the mechanical action of the chewing cycle.
